(19) United States
(12) Patent Application Publication
(10) Pub. No.: US 2006/0136018 A1
(43) Pub. Date: Jun. 22, 2006

(54) Apparatus for administering light stimulation

(76) Inventors: Leon Lack, Eden Hills (AU); Helen Wright, Teringie (AU)

Correspondence Address: Schwegman, Lundberg, Woessner & Kluth, P.A., P.O. Box 2938, Minneapolis, MN, 55402, US

(21) Appl. No.: 11/263,579

(22) Filed: Oct. 31, 2005

Related U.S. Application Data
(63) Continuation of application No. PCT/AU04/00559, filed on Apr. 30, 2004.

(30) Foreign Application Priority Data
May 1, 2003 (AU) 2003203967
Oct. 24, 2003 (AU) 2003905846

(51) Int. Cl. A61N 5/06

(52) U.S. Cl. 607/88

(57) ABSTRACT

An apparatus for administering light to effect re-timing of the human body clock is disclosed. In one form, the apparatus includes two pairs of light emitting diodes having an emission wavelength in the range 450 nm to 530 nm and a frame adapted to be worn on the face of a wearer. The frame is arranged to support the two pairs of light emitting diodes so that one pair of the light emitting diodes is supported adjacent a surface of each eye of the wearer. The light emitting diodes of each pair project a light output that illuminates a different area of the retina of a respective eye and are spaced apart so as to provide a viewing zone therebetween.

### RELATED APPLICATION

This application is a continuation under 35 U.S.C. 111(a) of PCT/AU2004/000559, filed Apr. 30, 2004, and published in English on Nov. 11, 2004 as WO 2004/096364 A1, which claims priority under 35 U.S.C. 119 to Australian Application Nos. 2003203967, filed May 1, 2003 and 2003905846, filed Oct. 24, 2003, which applications and publication are incorporated herein by reference.

### FIELD OF THE INVENTION

This invention relates to the presentation of relatively intense visual light stimulation for the treatment of circadian rhythm disorders including sleep onset insomnia and early morning awakening insomnia, jet-lag, shift work fatigue, winter depression or seasonal affective disorder (SAD), and other conditions requiring the re-timing of human circadian rhythms (24-hour body clock).

### BACKGROUND OF THE INVENTION

The timing of the body's circadian rhythms very strongly determines our best times to sleep and to be awake and alert. The anatomical pathways from the retina to the brain and the hormonal and biological mechanisms underlying this biological clock resetting capacity of bright light are well known. It was first demonstrated in 1980 that bright exposure could have a significant and direct impact on human physiological brain function. Since then, the use of bright light stimulation at appropriate times in the 24-hour period has been shown to change the timing of the body's endogenous circadian rhythms. In a normally entrained individual (for example, an individual who sleeps from 11 pm to 7 am) stimulation in the evening, up to about 4 am, delays circadian rhythms, whilst stimulation in the morning between 5 am and 9 am phase advances rhythms to an earlier time.

Inappropriate timing of circadian rhythms is implicated in several sleep/wake and mood disorders. Delayed and advanced sleep phase syndromes, which adversely affects the sleep and alertness of their sufferers for several hours each day, arise from delayed and advanced circadian rhythms. Research has shown that sleep onset insomnia and early morning awakening insomnia can be caused by delayed and advanced circadian rhythms respectively. Many patients with SAD have phase-delayed circadian rhythms.

Several methods have been proposed to deliver visual light stimulation for the adjustment of circadian rhythms. One approach utilises light boxes containing high intensity fluorescent light fixtures. However, these light boxes are large and require domestic high voltage power sources. Users must maintain visual contact with the light box when it is in use and as a consequence are restricted in their movement.

Another method is the use of a head mounted light visor as disclosed in U.S. Pat. No. 5,447,528. The light visor mounted at the forehead, well above the normal direction of gaze, is unlikely in most applied settings to stimulate the visual system with adequate light intensity to effect re-timing of the body clock. No study has confirmed their efficacy for this purpose.

The present invention seeks to provide an apparatus for administering sufficient intensity of an appropriate wavelength to effect re-timing of the human body clock, in a convenient and effective manner.

### SUMMARY OF THE INVENTION

The present invention provides an apparatus for administering light to effect re-timing of the human body clock including at least two light emitting diodes having an emission wavelength in the range 450 nm to 530 nm, and a frame adapted to be worn on the face of a wearer so as to support at least one of said light emitting diodes adjacent the surface of each eye to project light at the pupil of each eye.

The frame may be an eyeglass frame supported by the nose of the wearer in the customary manner. Any suitable eyeglass frame may be used to support each of said light emitting diodes adjacent the surface of a respective eye. One suitable eyeglass frame may include a pair of lens elements, each of which is supported between a respective ear stem and a nose-bridging member. The lens elements may be corrective or non-corrective lenses. Preferably, the eyeglass frame includes substantially conventional ear stems.

Preferably, the light emitting diodes are arranged so that the at least two light emitting diodes are powered by a single power supply (such as a battery). However, alternative arrangements are envisaged whereby a separate power supply is provided for each of the at least two light emitting diodes, or combinations of light emitting diodes. In either case, the power supply may be mechanically fitted to the frame, or alternatively, may be located remote from the frame but electrically connected to the at least two light emitting diodes using a suitable connectivity scheme.

In one embodiment, the light emitting diodes are part of an electrical circuit having terminals which allows the power supply to be electrically connected to the light emitting diodes using a suitable connectivity scheme. It is preferred that the frame itself form a part of the electrical circuit which is used to connect the power supply to the light emitting diodes. In this respect, the frame may include a first terminal 25 for connecting to a positive electrical potential of the power supply and a second terminal for connecting to a lower electrical potential of the power supply.

Preferably, the first and second terminals are provided on respective ear stems of the frame. Thus, in embodiments in which the frame is included as a part of the 30 electrical circuit, the ear stems may themselves be conductive.

The terminals may be connected to the power supply using a permanent joint (such as a solder joint) or using a make-break type electrical connector. Advantageously, the inclusion of the terminals on conductive ear stems provides a neat arrangement of a relatively simple design which avoids the need to include additional electrical wiring on the frame itself.

Each of the light emitting diodes are preferably terminated to one or more supporting members which also form a part of the electrical circuit between the first terminal and the second terminal of the frame. It is preferred that the at least two light emitting diodes are arranged so that each, or groups of, the light emitting diodes are terminated to a respective supporting member.

Each supporting member may include a substrate having conductive tracks arranged thereupon so as to provide a current path which includes the light emitting diodes terminated thereto and which extends between an input terminal and an output terminal of the substrate. Indeed, in one embodiment the substrate's terminals are connected to the frame so that the electrical circuit extends between the frame's first terminal to the frame's second terminal via the respective substrates and the nose-bridging member. Thus, according to this embodiment the nose-bridging member may also be electrically conductive.

In an embodiment, each substrate may be an elongate flexible substrate (such as a flexible printed circuit card) which is supported by the frame so as to lay across a surface of a respective lens element. In this way, the flexibility of the flexible substrate preferably allows the substrate to follow the curvature of the surface of the lens element. It is preferred that each flexible substrate lays across the back surface of a respective lens element.

Each substrate may be fitted to a respective lens element so as to be removable therefrom to facilitate repair or replacement of a defective or damaged component. However, in an alternative embodiment the substrate may be fitted to a respective lens element using an adhesive so that the substrate is permanently fixed to the lens element.

The legs (that is, the anode and cathode) of each light emitting diode may be terminated to a respective substrate and formed so as to locate the light emitting element of a respective light emitting diode apart from the substrate itself. It is preferred that each substrate be connected between a respective ear stem and the nose-bridging member. In this arrangement the each substrate will generally be located above the centre point of the respective monocular visual field. In this respect, reference to the term “monocular visual field” throughout this specification is to be understood to be reference to the extent of space that is visible to an eye as that eye fixates in a forward direction of gaze, and includes both central and peripheral vision. Moreover, reference to the term “wearer's visual field” throughout this specification is to be understood to be reference to the extent of space that is visible to both eyes of the wearer as each eye fixates in a forward direction of gaze, including both central and peripheral vision.

In an embodiment, the anode and cathode of the light emitting diodes are formed so as to depend downwardly from a respective substrate so as to locate the light emitting element of the light emitting diodes on or near a horizontal line that intersects the centre point of a respective monocular visual field of the wearer.

Preferably, two light emitting diodes are mounted onto eyeglass frames so that the light output of the light emitting diodes is aimed at the pupil of each eye. According to one embodiment, the two light emitting diodes are mounted in a spaced apart relationship so as to provide a viewing zone therebetween. Thus, the present invention also provides an apparatus for administering light to effect re-timing of the human body clock, the apparatus including:
two pairs of light emitting diodes having an emission wavelength in the range 450 nm to 530 nm; and
a frame adapted to be worn on the face of a wearer and arranged to support the two pairs of light emitting diodes so that one pair of the light emitting diodes is supported adjacent a surface of each eye of the wearer;
   wherein the light emitting diodes of each pair project a light output that illuminates a different area of the retina of a respective eye and wherein the light emitting diodes of each pair are spaced apart so as to provide a viewing zone therebetween.

In one embodiment, the different areas of the retina that are illuminated by each pair of diodes includes areas of the retina which are outside of the macula. Thus, in an embodiment the different areas are illuminated so as to exclude the macula. These different areas are located in what will subsequently be referred to in this specification as the “peripheral retina”. Moreover, for the purposes of this description, references to the term “exclude the macula”, when used in relation to the term “illumination of the different areas”, is to be understood as meaning that the macula is not directly illuminated by the light output of a respective light emitting diode pair. However, it will be appreciated that the macula may be indirectly illuminated by the light output of a respective light emitting diode pair as a result of intraocular scattering.

Illumination of areas of the retina which are exclude the macula is particularly beneficial because it avoids illumination of the macula itself. As will be appreciated, the macula is that part of the retina which is responsible for sharp straight-ahead vision (necessary for functions such as reading and driving a car). Thus, not illuminating the macula with the light output from the light emitting diodes reduces the extent to which the light output of the light emitting diodes interferes with normal vision. Moreover, because the peripheral retina contains a high expression of photoreceptors which govern circadian photoreception, the illumination of the peripheral retina is particularly advantageous for effecting re-timing of the human body clock.

In particular, a set of blue-light photoreceptors (the CRY1 and CRY2 cryptochromes) are localised in the inner nuclear layer (INL) and ganglion cell layer (GCL) of the peripheral retina. Accordingly, in one embodiment, light emitting diodes having a peak intensity at a wavelength of about 470 nm (that is, a wavelength to which the photoreceptors are responsive) are used. In this way, and when combined with a positioning of the light emitting diodes which allows different areas of the peripheral retina to be illuminated, photoreceptors which are specifically responsive to the light output of the light emitting diodes to effect re-timing of the human body clock are able to be illuminated.

Preferably, each light emitting diode projects a spreading beam shaped light output (such as a conical beam) and is supported adjacent a surface of each eye to provide an illuminated region on the surface thereof. Preferably, the illuminated region is a disc shaped region having an area which permits a portion of the light output to pass through the pupil of the respective eye so as to illuminate a respective area of the peripheral retina. In an embodiment, the disc shaped region may have a diameter that is substantially the same diameter as the diameter of the iris.

The supporting of the light emitting diodes adjacent to the surface of the eye combined with the geometry of the beam shaped light output may provide an illuminated region which permits areas of the peripheral retina to be illuminated throughout a range of movement of the eye.

In one embodiment, each light emitting diode is supported adjacent a surface of each eye and arranged so that the light output from each light emitting diode of a pair combines to provide a substantially uniform irradiance of substantially the entire area of the pupil of a respective eye. Indeed, in one embodiment the substantially uniform irradiance is provided so as to allow for a range of movement of the respective pupil. In this respect, reference to the term “a range of movement”, where used throughout this specification in relation to the pupil, is to be understood to be reference to an area within which the pupil may move as a result of dilation and/or rotation of the eye.

Preferably, the distance from the tips of the light emitting diodes to the corneal surface of the eyes is between 10 mm and 15 mm, and more preferably 12 mm. According to this embodiment, the disc shaped region preferably subtends between 15 degrees and 20 degrees. In a preferred arrangement, the light emitting diodes are positioned about 15 degrees away from the forward direction of gaze of the wearer both to the nasal and the lateral visual field.

In one embodiment, the effective intensity of the light emitting diodes at the cornea surface (measured photometrically) is in the range of 1000 lux to 4000 lux. More precisely, in terms of the electromagnetic energy incident at the pupil of the eye the effective intensity is preferably 100 uw/cm² to 400 uw/cm².

Preferably, the light emitting diodes are arranged such that the respective different areas of the peripheral retina which are illuminated by a pair of light emitting diodes includes a nasal area and a lateral area. Thus, in one embodiment, one light emitting diode from each pair is able to illuminate the nasal area of a respective peripheral retina and the other light emitting diode of that pair is able to illuminate the lateral area.

It is preferred that each viewing zone has a width in the range of 15 mm to 20 mm as measured between the light emitting diodes. Such an arrangement is particularly beneficial since it allows the light emitting diodes to project light via the pupil so as to illuminate areas of the peripheral retina which are not able to be illuminated when the light emitting diodes are spaced further apart, whilst also providing a viewing zone between the light emitting diodes which is adequate for viewing purposes.

The light emitting diodes from each pair are preferably arranged so that an external object that is visually obscured by a light emitting diode of one pair is not visually obscured by a light emitting diode of the other pair. In one embodiment, this is accomplished by arranging the pairs of light emitting diodes so that the viewing zones provided between each pair of light emitting diodes have different widths. Alternatively (or additionally), the pairs of light emitting diodes may themselves have different orientations so that light emitting diodes from different pairs are not located in corresponding visual field locations. Again, this arrangement may permit an external object obscured by the light emitting diode of one pair to not be obscured by a light emitting diode of the other pair.

In one embodiment, the pairs of light emitting diodes are arranged so that the light emitting diodes are collinear. It is preferred that the collinear arrangement of light emitting diodes be located along a horizontal axis which is located on, above or below the horizontal centreline of the wearer's monolocular visual field when the wearer adopts a forward direction of gaze (that is, when the wearer looks “straight ahead”).

When the light emitting diodes are located below the horizontal centreline of the wearer's monolocular visual field, the light emitting diodes may be arranged to project light at respective upper areas of the peripheral retina. Thus, in this embodiment one light emitting diode of each pair may illuminate an upper nasal area of the peripheral retina whilst the other light emitting diode may illuminate the upper lateral area of the peripheral retina. In this way, obstruction of the central portion of each monolocular visual field is reduced as compared to a case where the collinear arrangement of light emitting diodes are arranged on horizontal centreline 25 of the wearer's monolocular visual field.

In one embodiment, the collinear arrangement of the light emitting diodes are located about 15 degrees below the horizontal centreline of the wearer's monocular visual field. This arrangement has been found to provide a comfortable balance between effective illumination of the wearer's retina and reduction in obstruction of the wearer's monolocular visual field.

Where the light emitting diodes are located above the horizontal centreline of the wearer's normal monolocular visual field, the light emitting diodes may be arranged to project light at respective lower areas of the peripheral retina. Thus, according to this embodiment, one light emitting diode of each pair will illuminate a lower nasal area of the peripheral retina whilst the other light emitting diode will illuminate the lower lateral area of the peripheral retina.

Preferably, the pairs of light emitting diodes are spaced apart such that the distance between the midpoints of the viewing zones is substantially the same as the interpupillary distance of the wearer. Such an arrangement will generally result in the distance between the midpoints being in the range of 65 mm to 75 mm. It will be appreciated that the midpoint of each viewing zone may coincide with the centre point of the wearer's respective monocular visual field. However, it is also possible that the midpoint of one, or both, pair(s) of diodes may be horizontally offset relative to the wearer's normal monocular visual field so that the distance between the pairs is different than the interpupillary distance of the wearer. Indeed, in one embodiment the distance between the pairs is greater than the interpupillary distance of the wearer. Again, such an arrangement may permit an external object obscured by the light emitting diode of one pair to not be obscured by a light emitting diode of the other pair.

The present invention also provides an apparatus for administering light to effect re-timing of the human body clock, the apparatus including:
a light emitting element associated with each eye of a wearer, each light emitting element having an emission wavelength in the range 450 nm to 530 nm; and
a support member for supporting each light emitting element so that each light emitting element is arranged to illuminate an area of the retina of a respective eye;
   wherein the area of a respective retina that is illuminated by a respective light emitting element excludes the macula.

The present invention also provides an apparatus for administering light to effect re-timing of the human body clock, the apparatus including:
a light emitting element associated with each eye of a wearer, each light emitting element having an emission wavelength in the range 450 nm to 530 nm; and
a support member for supporting each light emitting element so that each light emitting element is arranged to illuminate an area of the retina of a respective eye;
   wherein the light emitting elements are supported so as to be located in non homologous areas of the wearer's visual field such that visual obstruction of the visual field of one eye caused by a respective light emitting element is not paired with visual obstruction of the visual field of the other eye caused by the other light emitting element so that binocular obstruction does not occur in any area of the wearer's visual field.

Some embodiments may include regulated electronic circuitry to deliver a constant low current (for example, 2 mA to 10 mA) from a 9 volt dry cell battery providing a constant current supply for up to 40 hours use.

Preferably, a low battery warning is provided when the battery is coming to the end of its life, by flashing of the light emitting diodes, indicating to the user that they should replace the battery.

At power turn on, there is preferably a ramping up of intensity over several seconds before the light emitting diodes reach the maximum intensity required for effective operation, thus allowing the user's eyes to adjust to the light slowly.

The present invention also provides a method of administering light to effect re-timing of the human body clock, the method including illuminating different areas of the peripheral retina of an eye of a wearer using a light emission having a wavelength in the range of 450 nm to 530 nm.

It will be apparent that because the apparatus is worn on the head, the spectacle mounted light emitting diodes ensure continued light stimulation irrespective of head movements. Additionally, being placed close to, and slightly below, the centre of the visual field of the wearer, the light emitting diodes ensure continued light simulation irrespective of gaze of the eyes or drooping of the eyelids.

### BRIEF DESCRIPTION OF THE DRAWINGS

Embodiments of the invention will now be described, by way of example only, with reference to the accompanying drawings in which:

FIG. 1 is a perspective view of an apparatus for administering light stimulation according to a first preferred embodiment of the invention;

FIG. 2 is a spectral power distribution for light emitting diodes that are suitable for use with the preferred embodiments of the invention;

FIG. 3 is a perspective view of an apparatus for administering light stimulation according to a second preferred embodiment of the invention;

FIG. 4 is a front view of the apparatus shown in FIG.3;

FIG. 5 is a rear view of the apparatus shown in FIG.3;

FIG. 6 is a front view of interaction between the light emitted by a pair of light emitting diodes of the apparatus shown in FIG.3 and an eye of a wearer;

FIG. 7 is a side view of interaction between the light emitted by a pair of light emitting diodes of the apparatus shown in FIG.3 and an eye of a wearer;

FIG. 8 is a top view showing the interaction between the light emitted by a pair of light emitting diodes of the apparatus shown in FIG.3 and an eye of a wearer;

FIG. 9 is a perspective view of an apparatus for administering light stimulation according to a third preferred embodiment of the invention; and

FIG. 10 is a perspective view of an apparatus for administering light stimulation according to a fourth preferred embodiment of the invention.

### DETAILED DESCRIPTION OF PREFERRED EMBODIMENTS OF THE INVENTION

### First Preferred Embodiment of the Invention.

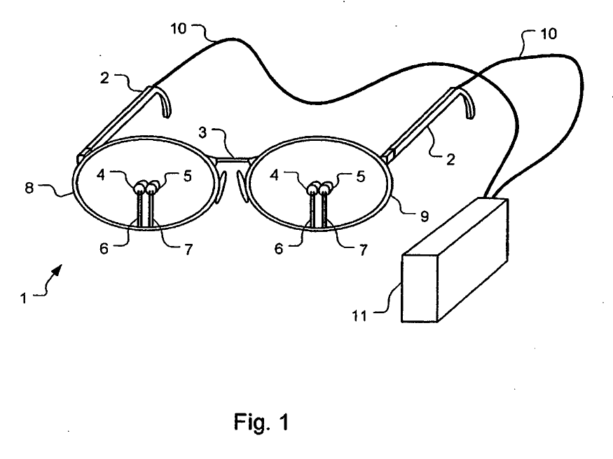

FIG. 1 shows an apparatus for administering light stimulation to effect re-timing of the human body clock according to a first preferred embodiment. In the illustrated embodiment, the apparatus takes the form of a substantially conventional eyeglass frame 1 having ear stems 2 and a nose bridge 3. The glasses are worn on the head of a patient in the conventional manner. Two light emitting elements, shown as light emitting diodes 4, 5, are mounted on bifurcated stems, 6, 7 to be located approximately central to the rims 8,0 of the eyeglass frame 1. The eyeglasses shown here are fitted with lenses. The light emitting diodes 4, 5 are aimed directly at the pupil of the adjacent eye and are positioned at about 12 mm from the eye surface. The light emitting diodes 4, 5 are a high intensity light emitting diode such as a Kingbright T-1 (3 mm) solid state diode that provides a dominant wavelength of about 470 nm.

As is shown, the light emitting diodes 4, 5 are powered through conventional wiring 10 connected with a power supply unit 11. The power supply unit 11 provides a current of about 3.5 milliamps from a 9 volt dry cell battery. This provides up to about 35 hours continuous use.

In tests leading to the present invention, it has been demonstrated that “blue” source (emission peak at 470 nm) high intensity light emitting diodes have biological effects on the timing of the biological clock. They have the effect of suppressing the production of melatonin from the pineal gland. In a direct test of re-timing capacity one administration of blue/green light emitting diodes light for two hours from midnight to 2 am in a applied setting resulted in an average phase delay of the melatonin circadian rhythm of 51 minutes. The same procedure with the white light emitting diodes resulted in an average phase delay of 30 minutes.

### Second Preferred Embodiment of the Invention.

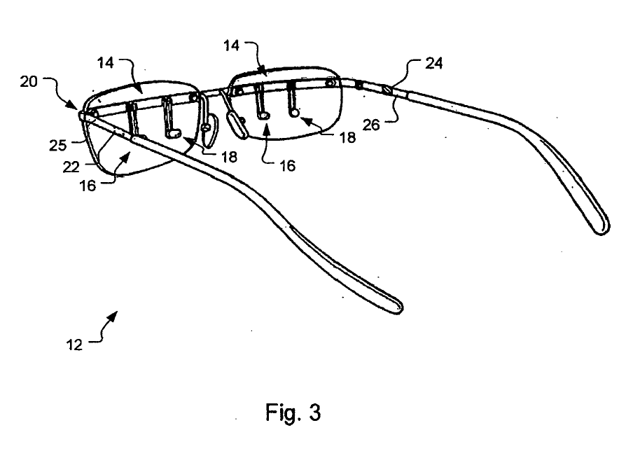

Referring to FIGS. 3 to 5 there is shown an apparatus 12 for administering light stimulation to effect re-timing of the human body clock according to a second preferred embodiment of the invention.

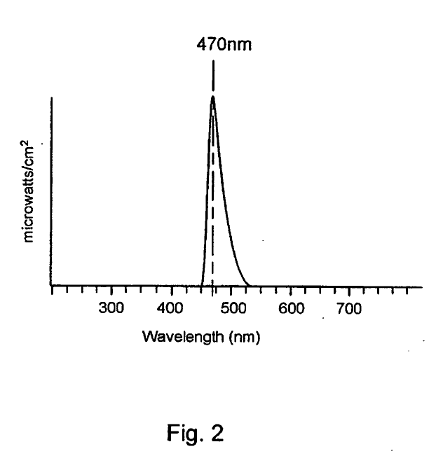

As is shown in FIG. 3, the apparatus 12 includes two pairs 14 of light emitting diodes 16, 18 and a frame 20 adapted to be worn on the face of a wearer so as to support each pair 14 of the light emitting diodes 16, 18 adjacent the surface of a respective eye to project light at the pupil of that eye. The light emitting diodes 16, 18 shown here are blue source colour type devices (such as Incan on Sic light emitting diodes) having a spectral power density similar to that shown in FIG. 2 and a viewing angle of about 20 degrees. As is shown, in FIG. 2 the light emitting diodes 16, 18 have a peak emission wavelength at around 470 nm. An emission wavelength of about 470 nm has been shown to be highly effective for effecting re-timing of the human body clock for both phase delaying and advancing the melatonin rhythm.

In use, and as will be described in more detail following, the light emitting diodes 16, 18 of a pair 14 illuminate different areas of the peripheral retina of the eye of the wearer.

As is shown in FIG. 3, the light emitting diodes 16, 18 of each pair 14 are spaced apart so as to provide a viewing zone 21 therebetween and arranged so as to project the light output from a respective light emitting diode at a respective area of the peripheral retina. As will be appreciated, the actual arrangement of the light emitting diodes 16, 18 will be dependent upon the positioning of the light emitting diodes 16, 18 relative to the wearer's eyes. In the illustrated embodiment, when the apparatus is fitted to the wearer, the light emitting diodes 16, 18 are positioned about 15 degrees away from the forward direction of gaze of the wearer both to the nasal and the lateral visual field.

Referring again to FIG. 3, the light emitting diodes 16, 18 are part of an electrical circuit having a first terminal 22 and a second terminal 24 which each allow a power supply (not shown) to be electrically connected to the light emitting diodes 16, 18 using a suitable connectivity scheme. The frame 20 shown here forms a part of the electrical circuit with the first terminal 22 and the second terminal 24 provided on respective ear stems 25, 26 of the frame 20. Thus, in the illustrated embodiment the ear stems 25, 26 are conductive.

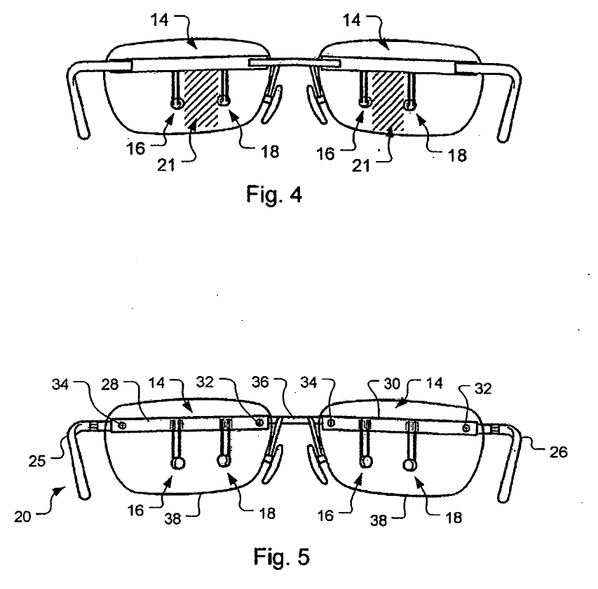

As is shown in FIG. 4, each pair 14 of light emitting diodes 16, 18 is terminated to a respective supporting member 28, 30. The supporting members 28, 30 also form a part of the electrical circuit between the first terminal 22 (ref. FIG. 3) and the second terminal 24 (ref. FIG. 3) of the frame 20 as well as providing mechanical support to the light emitting diodes 16, 18.

In the embodiment illustrated, each supporting member 28, 30 includes a substrate having conductive tracks (not shown) arranged thereupon so as to provide a current path which includes the light emitting diodes 16, 18 which are terminated thereto. This current path extends between an input terminal 32 and an output terminal 34 of each substrate 28, 30.

As is shown in FIG. 5, each substrate's 28, 30 terminals 32, 34 are connected to the frame 20 via a suitable mounting mechanism (shown here as fasteners) so that the electrical circuit extends between the frame's first terminal 22 (ref. FIG. 3) and the frame's second terminal 24 (ref. FIG. 3) via the respective substrates 28, 30 and conductive nose-bridging member 36. In the illustrated embodiment, each substrate 28, 30 is fitted to a respective lens element 38 so as to be removable to facilitate repair or replacement of a defective or damaged component.

In terms of the substrates 28, 30 themselves, in the illustrated embodiment each substrate 28, 30 is an elongate flexible substrate (shown here as a flexible printed circuit card) which is supported by the frame 20 so as to lay across a surface of a respective lens element 38. In this way, the flexibility of the substrate 28, 30 allows it to follow the curvature of the surface of the lens element 38. In the embodiment illustrated, each flexible substrate 28, 30 lays across the back surface of a respective lens element 38.

As is shown in FIG. 5, the legs (that is, the anode and cathode) of each light emitting diode 16, 18 are terminated to a respective substrate 28, 30 and formed so as to locate the light emitting element of a respective light emitting diode 16, 18 apart from the substrate 28, 30 itself.

Each substrate 28, 30 is connected between a respective ear stem 25, 26 and the conductive nose-bridging member 36. In the illustrated arrangement, each substrate 28, 30 will generally be located above the centre point of the normal monocular visual field of a respective eye of the wearer. Thus, the legs of the light emitting diodes 16, 18 have been formed so as to depend downwardly from a respective substrate 28, 30 so as to locate the light emitting element of each light emitting diode 16, 18 below a horizontal line which intersects the centre point of the respective monocular visual field. In this respect, it is preferred that the location of the light emitting elements of each light emitting diode be adjustable so as to adjust the width of the viewing zone and/or the position of the light emitting elements relative to the centre point of the monocular visual field of the wearer. Such adjustment advantageously allows the apparatus to be configured to suit the eye geometry of the wearer.

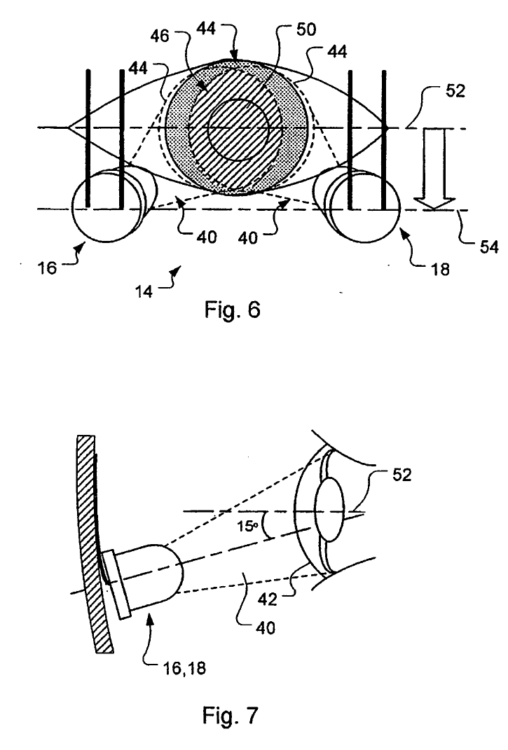

In terms of the positioning of the light emitting diodes 16, 18 relative to the wearer's eyes, as is shown in FIGS. 6 to 8, each light emitting diode 16, 18 is arranged to project a spreading beam shaped light output 40 (shown here as a conical beam) at the pupil of a respective eye.

As is shown in FIG. 6, each light emitting diode 16, 18 is supported adjacent a surface of each eye 42 so that the light output of each light emitting diode 16, 18 of a pair 14 illuminates an of area 44 of the wearer's eye. The area 44 shown in FIG. 5 are disc shaped regions 44 having an area that permits a portion of the light output of a respective light emitting diode 16, 18 to pass through the pupil 50 of the eye so as to illuminate a respective area of the peripheral retina. Indeed, each disc shaped region 44 has a diameter which is substantially the same diameter as the diameter of the iris 48. In this form, areas of the peripheral retina are able to be illuminated throughout a range of movement of the eye.

In the present case, the light emitting diodes 16, 18 are arranged so that the disc shaped regions 44 provided by each light emitting diode 16, 18 combine so as to provide a substantially uniform irradiance of substantially the entire area of the pupil 50 of a respective eye. In the illustrated embodiment, the combining of the disc shaped regions 44 provided by each light emitting diode 16, 18 occurs at the area 46 over which the disc shaped regions overlap. Advantageously, the area 46 allows for substantially uniform irradiance of substantially the entire area of the pupil 50 over a range of movement of the respective pupil 50.

In the illustrated embodiment, the pairs of light emitting diodes 14 shown are arranged so that the light emitting diodes 16, 18 are substantially collinear and located along an horizontal axis 54 which is itself located below the horizontal centreline 52 of the wearer's monolocular visual field.

Indeed, as is shown in FIG. 6, in the illustrated embodiment the collinear arrangement of the light emitting diodes 16, 18 is located about 15 degrees below the horizontal centreline 52 of the wearer's normal monocular visual field. This arrangement has been found to provide a comfortable balance between effective illumination of the wearer's retina and reduction in obstruction of the wearer's monolocular visual field.

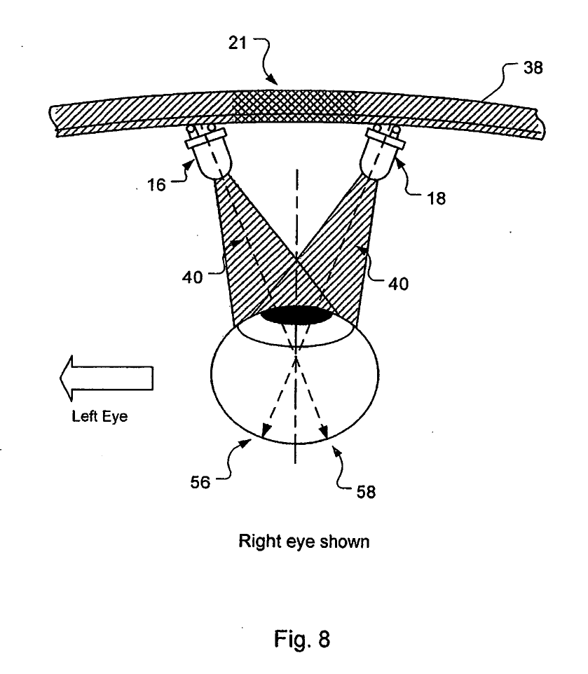

Turning now to FIG. 8, in the embodiment illustrated the light emitting diodes 16, 18 are arranged such that the areas of peripheral retina which are able to be illuminated include a nasal area 56 and a lateral area 58. That is to say, one light emitting diode 18 from each pair is able to illuminate the nasal area 56 of a respective peripheral retina, whilst the other light emitting diode 16 is able to illuminate the lateral area 58.

Returning again to FIG. 6, in the illustrated embodiment the light emitting diodes 16, 18 are located below the centreline 52 of the wearer's normal monolocular visual field so as to project light at respective upper areas of the peripheral retina. Thus, one light emitting diode of each pair illuminates an upper nasal area of the peripheral retina, whilst the other light emitting diode illuminates the upper lateral area of the peripheral retina.

The viewing zone 21 located between the light emitting diodes 16, 18 has a width in the range of 15 mm to 20 mm. As a result of this spacing, the light emitting diodes 16, 18 are able to illuminate areas of the peripheral retina which are not able to be illuminated when the light emitting diodes 16, 18 are spaced further apart. This spacing provides a viewing zone 21 between the light emitting diodes 16, 18 of each pair which provides 30 degrees to 40 degrees of central visual field free of visual obstruction. Moreover, the pairs of light emitting diodes 16, 18 are spaced apart such that the distance between the midpoints of each viewing zone 21 is substantially the same as the interpupillary distance of the wearer so that the mid-point of each viewing zone 21 coincides with the centre point of the wearer's normal monocular visual field.

It is possible that the midpoint of one, or both, pair(s) of light emitting diodes may be horizontally offset relative to the respective monocular visual field so that the distance between the pairs is different than the interpupillary distance of the wearer. Indeed, in one embodiment the distance between the pairs is greater than the interpupillary distance of the wearer.

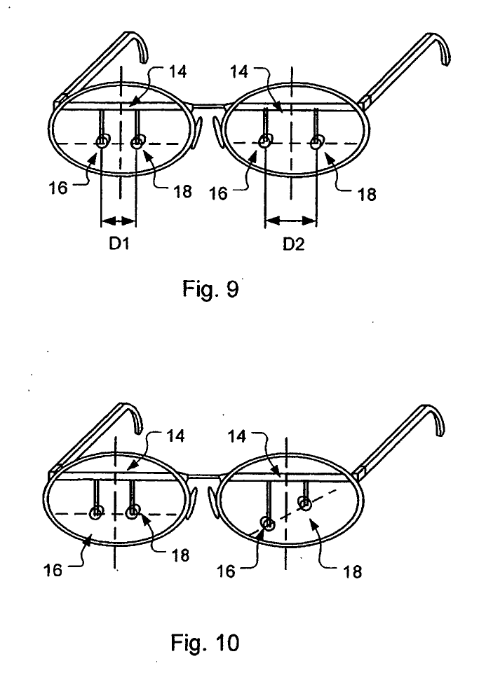

As is shown in FIGS. 9 and 10, the light emitting diodes may be supported so as to be located in non homologous areas of the wearer's visual field such that visual obstruction of the monocular visual field of one eye caused by a respective light emitting diode pair 14 is not paired with visual obstruction of the monocular visual field of the other eye caused by the other light emitting diode pair 14 so that binocular obstruction does not occur in any area of the wearer's visual field.

As is shown in FIG. 9, in one embodiment in which light emitting diodes are supported so as to be located in non homologous areas of the wearer's visual field, the light emitting diodes are arranged so that the light emitting diodes 16, 18 from each pair 14 provide viewing zones 21 having different widths D1, D2. It is envisaged that such an arrangement will allow an external object visually obscured by the light emitting diode of one pair to not be visually obscured by a light emitting diode of the other pair.

As is shown in FIG. 10, in an alternative embodiment the locating of the light emitting diodes 16, 18 in non homologous areas of the wearer's visual field includes arranging the pairs 14 of light emitting diodes 16, 18 so that the pairs 14 have different orientations relative to one another

The foregoing describes only two preferred embodiments of the invention and it will be appreciated that modifications can be made without departing from the scope of the invention.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. An apparatus for administering light to effect re-timing of the human body clock including at least two light emitting diodes having an emission wavelength in the range 450 nm to 530 nm, a frame adapted to be worn on the face of a wearer and support one of said light emitting diodes adjacent the surface of each eye to direct light at the pupil of each eye.

2. An apparatus according to claim 1, wherein the frame is in the form of an eyeglass frame supported by a nose in the customary manner.

3. An apparatus according to claim 2, wherein the eyeglass frame includes substantially conventional ear stems.

4. An apparatus according to claim 1, wherein the light emitting diodes have a peak intensity at a wavelength of about 470 nm

5. An apparatus according to claim 1 wherein the light emitting diodes project a light output that illuminates an area of the retina of a respective eye, the area excluding the macula.

6. An apparatus according to claim 2, wherein a pair of the light emitting diodes are each mounted at a bottom branch of a respective eye frame of the eyeglass such that the light output of each light emitting diode is aimed at the pupil of a respective eye.

7. An apparatus according to claim 6 wherein each light emitting diode of a pair projects a light output that illuminates a different area of the retina of a respective eye and wherein the light emitting diodes of each pair are spaced apart so as to provide a viewing zone therebetween.

8. An apparatus according to claim 7 wherein each viewing zone has a width that is substantially the same as the diameter of the iris of a respective eye.

9. An apparatus according to claim 1 wherein each light emitting diode is arranged so that the respective light output provides a substantially uniform irradiance of substantially the entire area of the pupil of a respective eye.

10. An apparatus according to claim 9 wherein the substantially uniform irradiance is provided over a range of movement of the respective pupil.

11. An apparatus according to claim 1 wherein the light emitting diodes are positioned 10 to 20 degrees below a line that intersects the centrepoints of the monocular visual fields of the eyes of the wearer.

12. An apparatus according to claim 6 wherein the light emitting diodes are supported so as to be located in non homologous areas of the wearer's visual field such that visual obstruction of the monocular visual field of one eye caused by a respective light emitting diode pair is not paired with visual obstruction of the monocular visual field of the other eye caused by the other light emitting diode pair so that binocular obstruction does not occur in any area of the wearer's visual field.

13. The apparatus as claimed claim 1, wherein the distance from the tips of the light emitting diodes to the corneal surface of respective eyes is between substantially 10 mm to 15 mm.

14. The apparatus as claimed in claim 13, wherein the distance from the tips of the light emitting diodes to the corneal surface of respective eyes is substantially 12 mm.

15. The apparatus as claimed in claim 1, wherein the effective intensity of the light emitting diodes at a corneal surface is in the range of 1000 lux to 4000 lux.

16. The apparatus as claimed in claim 1, further including electronic circuitry to deliver a constant low current from a battery.

17. The apparatus as claimed in any one of claims 1, wherein the electronic circuitry includes the frame.

18. The apparatus as claimed in claim 16, wherein a low battery warning is provided when the battery is coming to the end of its life.

19. The apparatus as claimed in claim 18, wherein the low battery warning is provided by flashing of the light emitting diodes.

20. The apparatus as claimed in claim 1, wherein there is a ramping up of intensity over substantially 5 seconds before the light emitting diodes reach a maximum intensity required for effective operation, thus allowing a user's eyes to adjust to the light slowly.

21. An apparatus for administering light to effect re-timing of the human body clock, the apparatus including:
two pairs of light emitting diodes having an emission wavelength in the range 450 nm to 530 nm; and
a frame adapted to be worn on the face of a wearer and arranged to support the two pairs of light emitting diodes so that one pair of the light emitting diodes is supported adjacent a surface of each eye of the wearer;
wherein the light emitting diodes of each pair project a light output that illuminates a different area of the retina of a respective eye and wherein the light emitting diodes of each pair are spaced apart so as to provide a viewing zone therebetween.

22. An apparatus according to claim 21 wherein the different areas of a respective retina include areas of the peripheral retina, the different areas excluding the macula.

23. An apparatus according to claim 21 wherein the light emitting diodes have a peak intensity at a wavelength of about 470 nm.

24. An apparatus according to claim 21 wherein each light emitting diode is supported adjacent a surface of each eye and arranged so that the light output from each light emitting diode of a pair combine to provide a substantially uniform irradiance of substantially the entire area of the pupil of a respective eye.

25. An apparatus according to claim 24 wherein the substantially uniform irradiance is provided over a range of movement of the respective pupil.

26. An apparatus according to claim 21 wherein each light emitting diode pair is positioned 10 to 20 degrees below the centre of the monocular visual field of a respective eye.

27. An apparatus according to claim 21 wherein the light emitting diodes provide an effective intensity at a corneal surface of a respective eye in the range of 1000 lux to 4000 lux.

28. An apparatus according to claim 21 wherein the electromagnetic energy incident at the pupil of the eye is in the range of 100 uw/cm² to 400 uw/cm².

29. An apparatus according to claim 22 wherein the light emitting diodes are arranged so that the respective different areas of the peripheral retina which are illuminated by a pair of light emitting diodes includes a nasal area and a lateral area.

30. An apparatus according to claim 21 wherein each viewing zone has a width that is substantially the same as the diameter of the iris of a respective eye.

31. An apparatus according to claim 21 wherein the light emitting diodes are supported so as to be located in non homologous areas of the wearer's visual field such that visual obstruction of the monocular visual field of one eye caused by a respective light emitting diode pair is not paired with visual obstruction of the monocular visual field of the other eye caused by the other light emitting diode pair so that binocular obstruction does not occur in any area of the wearer's visual field.

32. An apparatus according to claim 31 wherein the locating of the light emitting diodes in non homologous areas of the wearer's visual field includes arranging the pairs of light emitting diodes so that the viewing zones provided between each pair of light emitting diodes have different widths.

33. An apparatus according to claim 32 wherein the locating of the light emitting diodes in non homologous areas of the wearer's visual field includes arranging the pairs of light emitting diodes so that the pairs have different orientations relative to one another.

34. An apparatus according to claim 21 wherein the pairs of light emitting diodes are arranged so that the light emitting diodes are located on a horizontal axis which is located on or below the horizontal centreline of the wearer's monocular visual field.

35. An apparatus according to claim 34 wherein the light emitting diodes are located on a horizontal axis which is about 15 degrees below the horizontal centreline of the wearer's monocular visual field.

36. An apparatus according to claim 21 wherein the light emitting diodes of each pair are spaced apart such that a distance between a midpoint of each viewing zone is substantially the same as the interpupillary distance of the wearer.

37. An apparatus according to claim 21 wherein each viewing zone has a midpoint that lies on a vertical centreline that intersects with the centre point of the respective monocular visual field.

38. An apparatus for administering light to effect re-timing of the human body clock, the apparatus including:
a light emitting element associated with each eye of a wearer, each light emitting element having an emission wavelength in the range 450 nm to 530 nm; and
a support member for supporting each light emitting element so that each light emitting element is arranged to illuminate an area of the retina of a respective eye;
wherein the area of a respective retina that is illuminated by a respective light emitting element excludes the macula.

39. An apparatus according to claim 38 wherein each light emitting element is arranged so as to provide a substantially uniform irradiance of substantially the entire area of the pupil of a respective eye

40. An apparatus according to claim 39 wherein the substantially uniform irradiance is provided over a range of movement of the respective pupil.

41. An apparatus according to claim 38 wherein the light emitting elements are supported so as to be located in non homologous areas of the wearer's visual field such that visual obstruction of one ocular field caused by a respective light emitting element is not paired with visual obstruction of the other ocular field caused by the other light emitting element so that binocular obstruction does not occur in any area of the wearer's visual field.

42. An apparatus for administering light to effect re-timing of the human body clock, the apparatus including:
a light emitting element associated with each eye of a wearer, each light emitting element having an emission wavelength in the range 450 nm to 530 nm; and
a support member for supporting each light emitting element so that each light emitting element is arranged to illuminate an area of the retina of a respective eye;
wherein the light emitting elements are supported so as to be located in non homologous areas of the wearer's visual field such that visual obstruction of the monocular visual field of one eye caused by a respective light emitting element is not paired with visual obstruction of the monocular visual field of the other eye caused by the other light emitting element so that binocular obstruction does not occur in any area of the wearer's visual field.

43. An apparatus according to claim 42 wherein the area of a respective retina that is illuminated by a respective light emitting element excludes the macula.

44. An apparatus according to claim 42 wherein each light emitting element is arranged so as to provide a substantially uniform irradiance of substantially the entire area of the pupil of a respective eye.

45. An apparatus according to claim 44 wherein the uniform irradiance is provided over a range of movement of the respective pupil.

46. An apparatus for administering light to effect re-timing of the human body clock, the apparatus including:
two pairs of light emitting elements having an emission wavelength in the range 450 nm to 530 nm; and
a frame adapted to be worn on the face of a wearer and arranged to support the two pairs of light emitting elements so that one pair of the light emitting elements is supported adjacent a surface of each eye of the wearer such that the light emitting elements of each pair project a light output that illuminates different areas of the peripheral retina;
wherein the light emitting elements of each pair are spaced apart so as to provide a viewing zone therebetween.

47. An apparatus according to claim 46 wherein different areas of the peripheral retina include a nasal area and a lateral area of the peripheral retina.

48. A method of administering light to effect re-timing of the human body clock, the method including illuminating different areas of the peripheral retina of an eye of a wearer using a light emission having a wavelength in the range of 450 nm to 530 nm.

49. A method of administering light to effect re-timing of the human body clock, the method including using an apparatus as claimed in claim 1.
